SEQUENCE LISTING (6 sequences)

SEQ ID NO: 1 - DNA, 1924 bp, Homo sapiens
cgcccgggcaggttagaattcagcgaccgcttaattctaggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggtcttgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgaccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgagcatctcttctccataagatagtgtgataaacacggtcatgaataaagttattttccccgaaaaaaaaaaaaaaaaaaaaaaaaaa

SEQ ID NO: 2 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWSCFNDSNICLVSWEDIQCTYGNPNYHWQETAYLLVYMKMEC

SEQ ID NO: 3 - DNA, 3330 bp, Homo sapiens
cttttcttttctccaggaagagagctgtgaccagcagcgtcccttattcgcttggccttggttcctgtttgcactggctacagcagggcactggcccctactgtcaccgccacctacacaaagaccctatctctgagcgctgcagcctactgttcagccccaggtttgaggatggatgccctggacgcttcgaagctactggatgaggagctgtattcaagacagctgtatgtgctgggctcacctgccatgcagaggattcagggagccagggtcctggtgtcaggcctgcagggcctgggggccgaggtggccaagaacttggttctgatgggtgtgggcagcctcactctgcatgatccccaccccacctgctggtccgacctggctgcccagtttctcctctcagagcaggacttggaaaggagcagagccgaggcctctcaagagctcttggctcagctcaacagagctgtccaggtcgtcgtgcacacgggtgacatcactgaggacctgctgttggacttccaggtggtggtgctgactgctgcaaagctggaggagcagctgaaggtgggcaccttgtgtcataagcatggagtttgctttctggcggctgacacccggggcctcgtggggcagttgttctgtgactttggtgaggacttcactgtgcaggaccccacagaggcagaacccctgacagctgccatccagcacatctcccagggctcccctggcattctcactctgaggaaaggggccaatacccactacttccgtgatggagacttggtgactttctcgggaattgagggaatggttgagctcaacgactgtgatccccggtctatccacgtgcgggaggatgggtccctggagattggagacacaacaactttctctcggtacttgcgtggtggggctatcactgaagtcaagagacccaagactgtgagacataagtccctggacacagccctgctccagccccatgtggtggcccagagctcccaggaagttcaccatgcccactgcctgcatcaggccttctgtgcactgcacaagttccagcacctccatggccggccaccccagccctgggatcctgttgatgcagagactgtggtgggcctggcccgggacctggaaccactgaagcggacagaggaagagccactggaagagccactggatgaggccctagtgcggacagtcgccctaagcagtgcaggtgtcttgagccctatggtggccatgctgggtgcagtagctgcccaggaagtgctgaaggcaatctccaggaagttcatgcctctggaccagtggctttactttgatgccctcgattgtcttccggaagatggggagctccttcccagtcctgaggactgtgccctgagaggcagccgctatgatgggcaaattgcagtgtttggggctggttttcaggagaaactgagacgccagcactacctcctggtgggcgctggtgccattggttgtgagctgctcaaagtctttgccctagtgggactgggggccgggaacagcgggggcttgactgttgttgacatggaccacatagagcgctccaatctcagccgtcagttcctcttcaggtcccaggacgttggtagacccaaggcagaggtggctgcagcagctgcccggggcctgaacccagacttacaggtgatcccgctcacctacccactggatcccaccacagagcacatctatggggataactttttctcccgtgtggatggtgtggctgctgccctggacagtttccaggcccggcgctatgtggctgctcgttgcacccactatctgaagccactgctggaggcaggcacatcgggcacctggggcagtgctacagtattcatgccacatgtgactgaggcctacagagcccctgcctcagctgcagcttctgaggatgccccctaccctgtctgtaccgtgcggtacttccctagcacagccgagcacaccctgcagtgggcccggcatgagtttgaagaactcttccgactgtctgcagagaccatcaaccaccaccaacaggcacacacttccctggcagacatggatgagccacagacactcaccttactgaagccagtgcttggggtcctgagagtgcgtccacagaactggcaagactgtgtggcgtgggctcttggccactggaaactctgctttcattatggcatcaaacagctgctgaggcacttcccacctaataaagtgcttgaggatggaactcccttctggtcaggtcccaaacagtgtccccagcccttggagtttgacaccaaccaagacacacacctcctctacgtactggcagctgccaacctgtatgcccagatgcatgggctgcctggctcacaggactggactgcactcagggagctgctgaagctgctgccacagcctgacccccaacagatggcccccatctttgctagtaatctagagctggcttcggcttctgctgagtttggccctgagcagcagaaggaactgaacaaagccctggaagtctggagtgtgggccctcccctgaagcctctgatgtttgagaaggatgatgacagcaacttccatgtggactttgtggtagcggcagctagcctgagatgtcagaactacgggattccaccggtcaaccgtgcccagagcaagcgaattgtgggccagattatcccagccattgccaccactacagcagctgtggcaggcctgttgggcctggagctgtataaggtggtgagtgggccacggcctcgtagtgcctttcgccacagctacctacatctggctgaaaactacctcatccgctatatgccttttgccccagccatccagacgttccatcacctgaagtggacctcttgggaccgtctgaaggtaccagctgggcagcctgagaggaccctggagtcgctgctggctcatcttcaggagcagcacgggttgagggtgaggatcctgctgcacggctcagccctgctctatgcggccggatggtcacctgaaaagcaggcccagcacctgcccctcagggtgacagaactggttcagcagctgacaggccaggcacctgctcctgggcagcgggtgttggtgctagagctgagctgtgagggtgacgacgaggacactgccttcccacctctgcactatgagctgtgacaaggcagccaccctgtcacctagctcaatggagccccggatcccaagccctgcattgtaagcccacagtaggcactcaataaatgcttgttaaaggaaggcaaaaaaaaaaaaaaaaaa

SEQ ID NO: 4 - protein, 1012 aa, Homo sapiens
MDALDASKLLDEELYSRQLYVLGSPAMQRIQGARVLVSGLQGLGAEVAKNLVLMGVGSLTLHDPHPTCWSDLAAQFLLSEQDLERSRAEASQELLAQLNRAVQVVVHTGDITEDLLLDFQVVVLTAAKLEEQLKVGTLCHKHGVCFLAADTRGLVGQLFCDFGEDFTVQDPTEAEPLTAAIQHISQGSPGILTLRKGANTHYFRDGDLVTFSGIEGMVELNDCDPRSIHVREDGSLEIGDTTTFSRYLRGGAITEVKRPKTVRHKSLDTALLQPHVVAQSSQEVHHAHCLHQAFCALHKFQHLHGRPPQPWDPVDAETVVGLARDLEPLKRTEEEPLEEPLDEALVRTVALSSAGVLSPMVAMLGAVAAQEVLKAISRKFMPLDQWLYFDALDCLPEDGELLPSPEDCALRGSRYDGQIAVFGAGFQEKLRRQHYLLVGAGAIGCELLKVFALVGLGAGNSGGLTVVDMDHIERSNLSRQFLFRSQDVGRPKAEVAAAAARGLNPDLQVIPLTYPLDPTTEHIYGDNFFSRVDGVAAALDSFQARRYVAARCTHYLKPLLEAGTSGTWGSATVFMPHVTEAYRAPASAAASEDAPYPVCTVRYFPSTAEHTLQWARHEFEELFRLSAETINHHQQAHTSLADMDEPQTLTLLKPVLGVLRVRPQNWQDCVAWALGHWKLCFHYGIKQLLRHFPPNKVLEDGTPFWSGPKQCPQPLEFDTNQDTHLLYVLAAANLYAQMHGLPGSQDWTALRELLKLLPQPDPQQMAPIFASNLELASASAEFGPEQQKELNKALEVWSVGPPLKPLMFEKDDDSNFHVDFVVAAASLRCQNYGIPPVNRAQSKRIVGQIIPAIATTTAAVAGLLGLELYKVVSGPRPRSAFRHSYLHLAENYLIRYMPFAPAIQTFHHLKWTSWDRLKVPAGQPERTLESLLAHLQEQHGLRVRILLHGSALLYAAGWSPEKQAQHLPLRVTELVQQLTGQAPAPGQRVLVLELSCEGDDEDTAFPPLHYEL

SEQ ID NO: 5 - DNA, 498 bp, Homo sapiens
atgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgaacatcctggtgaggaataacaagggccgcagcagcacctacgaggtgcggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaa

SEQ ID NO: 6 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLNILVRNNKGRSSTYEVRLTQTVAHLKQQVSGLEGVQDDLFWLTFEGKPLEDQLPLGEYGLKPLSTVFMNLRLRGGGTEPGGRS

(19) United States
(12) Patent Application Publication
(10) Pub. No.: US 2009/0136479 A1
(43) Pub. Date: May 28, 2009

(54) Ubiquitin-like protein isg15 for hiv inhibition

(75) Inventors: Paula M. Pitha-Rowe, Baltimore, MD (US); Atsushi Okumura, Cockeysville, MD (US); Myriam S. Kunzi, Annandale, VA (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/630,544

(22) PCT Filed: Jun. 28, 2005
(86) PCT No.: PCT/US05/23237

§ 371 Date: Oct. 17, 2007

Related U.S. Application Data
(60) Provisional application No. 60/583,584, filed on Jun. 28, 2004.

(51) Int. Cl. A61K 39/395; C12N 5/06; C12N 15/00

(52) U.S. Cl. 424/130.1; 435/375; 435/320.1

(57) ABSTRACT

The instant invention provides methods for the treatment and prevention of viral infection, e.g., HIV infection, based on the discovery that viral replication utilizes the ubiquitination pathway of the host cell to replicate. Specifically, the invention provides methods for the treatment and prevention of viral infection, e.g., HIV infection, by modulation of ISG15 conjugation and deconjugation, e.g., by modulation of the activity or expression of UBP43 or UBEL-1.

### RELATED APPLICATIONS

This application claims the benefit of U.S. Provisional Application No. 60/583,584, filed Jun. 28, 2005, the entire contents of which are incorporated herein by reference.

### GOVERNMENT SPONSORED RESEARCH

The present invention was made with government support under Grant No. 1R21AI054276, awarded by the National Institutes of Health. Accordingly, the Government has certain rights in this invention.

### BACKGROUND OF THE INVENTION

The type-1 human immunodeficiency virus (HIV-1) has been implicated as the primary cause of the slowly degenerate disease of the immune system termed acquired immune deficiency syndrome (AIDS) (Barre-Sinoussi, F. et al., 1983 Science 220:868-70; Gallo, R. et al. 1984, Science 224:500-3). Infection of the CD4+ subclass of T-lymphocytes with the HIV-1 virus leads to depletion of this essential lymphocyte subclass which inevitably leads to opportunistic infections, neurological disease, neoplastic growth and eventually death. HIV-1 infection and HW-1 associated diseases represent a major health problem and considerable attention is currently being directed towards the successful design of effective therapeutics.

HIV-1 is a member of the lentivirus family of retroviruses (Teich, N. et al., 1984 In RNA Tumor Viruses ed. R. Weiss, N. Teich, H. Varmus, J. Coffin CSH Press, pp. 949-56). The life cycle of HI-1 is characterized by a period of proviral latency followed by active replication of the virus. The primary cellular target for the infectious HIV-1 virus is the CD4 subset of human T-lymphocytes.

After binding to the cell surface, the HIV-1 virion becomes internalized, and once inside the cell, the viral life cycle begins by conversion of the RNA genome into linear DNA molecules. This process is dependent on the action of the virally encoded reverse transcriptase. Following replication of the viral genome, the linear DNA molecule integrates into the host genome through the action of the viral integrase protein, thus establishing the proviral form of HIV-1.

During the early phase of proviral expression, transcription of the viral genome results in expression of regulatory proteins such as Tat, Nef and Rev. Transcriptional activation of the proviral DNA is mediated through the viral 5′ LTR sequences (long terminal repeats). The initial low level of viral transcription is dramatically increased by the HIV encoded transactivator protein termed tat (transactivator protein) (Cullen, B. R. et al. 1989, Cell 58:423-26). The Rev protein promotes the transition from the early phase expression of regulatory proteins to late phase expression of structural proteins. Assembly of newly synthesized viral particles is followed by budding of virus particles from the cell membrane allowing the virus to infect new cells.

The HIV-1 virus is capable of establishing a latent state of infection for prolonged periods of time. Individuals infected with the human immunodeficiency virus may remain clinically healthy for long periods of time, with the estimated average length of the asymptomatic period between primary HIV infection and the progression to AIDS and increase in viral replication being approximately 8 to 10 years. Several possibilities have been proposed to explain the maintenance of the low levels of viral replication during this period of latency. It is generally believed that the humoral immune response to HIV-1 is not sufficiently protective against progression of the disease and attention has, therefore, turned to the possibility that the T-lymphocyte population of cells, e.g., cytotoxic T cells, may play a major role in inhibition of HIV-1 replication.

Several biological processes are controlled by the ubiquitination of cellular protein. Cellular processes that are affected by ubiquitin modification include the regulation of gene expression, regulation of the cell cycle and cell division, cellular housekeeping, cell-specific metabolic pathways, disposal of mutated or post-translationally damaged proteins, the cellular stress response, modification of cell surface receptors, DNA repair, import of proteins into mitochondria, uptake of precursors into neurons, biogenesis of mitochondria, ribosomes, and peroxisomes, apoptosis, and growth factor-mediated signal transduction.

Previous studies have demonstrated the role of a ubiquitin-like protein, ISG15, in viral replication (Kunzi, et al. (1996) Journal of Interferon and Cytokine Research 16:919-927). However, the mechanism of ISG15-mediated antiviral action has not been elucidated.

### SUMMARY OF THE INVENTION

The instant invention is based on the discovery that an ubiquitin-like protein named ISG15 (Accession No.: AAN86983) interferes with the ubiquitin pathway exploited by HIV-1 during its replication cycle. ISG15 is a component of the innate 5 antiviral response in mammals and it is induced by interferon and viral infection. Since ubiquitin conjugation is critical for HIV-1 replication, the regulation of ISG15 provides a novel target for the inhibition of viral replication. ISG15 mediated inhibition is dependant on conjugation of ISG15 to cellular proteins. An increase in ISG15 conjugation increases the activity of ISG15 by the ISG15 activating enzyme UBEL-1 (also called UBEIL herein and in the literature) (Accession No. NP_{—}003326) (Kok, K. et al. (1993) Proc. Natl. Acad. Sci. U.S.A. 90:6071-6075) and increases the ISG15 mediated inhibition of HIV-1 replication. ISG15 decojugating enzyme UBP43 (Accession No. CAB76398) (Schwer, H. et al. (2000) Genomics 65:44-52) negates the inhibitory effect. ISG15 conjugation is critical for the inhibition of HIV-1 replication and, therefore, modulation of ISG15 expression and/or activity and its conjugation to cellular proteins provides a novel target for inhibition of viral replication.

Accordingly, the instant invention provides methods and compositions for the treatment and prevention of viral replication, e.g., of viruses which use the ubiquitin mediated endosomal pathway such as HIV and Ebola virus.

In one embodiment, the instant invention provides a method of inhibiting the replication of a virus by contacting a cell infected with a virus with a compound that, modulates the activity or expression of a polypeptide involved in the ubiquitination pathway used during viral replication, thereby inhibiting the replication of the virus.

In a related embodiment, the modulation of the activity or expression of a polypeptide involved in the ubiquitination pathway leads to increased conjugation of ISG15 to cellular proteins involved in viral replication.

In a specific embodiment, the modulation is the upregulation of the activity or expression of, for example, UBEL-1. In another specific embodiment, the modulation is the downregulation of the expression or activity of an ISG15 deconjugating enzymes (ISG-DCE), e.g., UBP43.

In a related embodiment, the virus is selected from one or more retroviruses, e.g., human immunodeficiency virus, human T-cell leukemia virus type 1, and human T-cell leukemia virus type 2 or Class A viruses, e.g., filovirus (more commonly known as the Ebola virus).

In another embodiment, the activity is modulated by a molecule selected from the group consisting of a small molecule, a peptide, a peptide mimetic, a lipid, a lipid, and an antibody. In another embodiment, the expression is modulated by siRNA, a ribozyme, or antisense RNA.

In specific embodiments, the virus is drug sensitive or drug resistant.

In another aspect, the instant invention provides a method of inhibiting the replication of viruses that rely on the cellular endosomal pathways to replicate by contacting a cell infected with the virus with a compound capable of inhibiting the activity or expression of UBP43, thereby inhibiting viral replication.

In a related embodiment, the inhibition of the expression or activity of UBP43 leads to inhibition of the deconjugation of ISG15 from proteins involved in HIV replication.

In a related embodiment, the virus is selected from one or more retroviruses, e.g., human immunodeficiency virus, human T-cell leukemia virus type 1, and human T-cell leukemia virus type 2 or Class A viruses, e.g., filovirus (more commonly known as the Ebola virus).

In another embodiment, the activity of UBP43 is modulated by a molecule selected from the group consisting of a small molecule, a peptide, a peptide mimetic, a lipid, and an antibody. In another embodiment, the expression of UBP43 is modulated by siRNA, a ribozyme, or antisense RNA.

In specific embodiments, the virus is drug sensitive or drug resistant.

In another aspect, the instant invention provides a method of inhibiting the replication of HIV by contacting a cell infected with HIV with a compound capable of inhibiting the activity or expression of UBP43, thereby inhibiting HIV replication.

In a related embodiment, the inhibition of the expression or activity of UBP43 leads to inhibition of the deconjugation of ISG15 from proteins involved in HIV replication.

In another embodiment, the activity of UBP43 is modulated by a molecule selected from the group consisting of a small molecule, a peptide, a peptide mimetic, a lipid, and an antibody. In another embodiment, the expression of UBP43 is modulated by siRNA, a ribozyme, or antisense RNA.

In specific embodiments, the virus is drug sensitive or drug resistant.

In yet another aspect, the invention provides a method of inhibiting the replication of viruses that rely on the cellular endosomal pathways to replicate by contacting a cell infected with the virus with a compound capable of stimulating the expression or activity of UBEL-1, thereby inhibiting viral replication.

In a related embodiment, stimulating the expression or activity of UBEL-1 leads to increased conjugation of ISG15 to proteins involved in viral replication. In a related embodiment, the expression is stimulated by gene therapy.

In specific embodiments, the virus is drug sensitive or drug resistant.

In another aspect, the invention provides a method of inhibiting the expression of UBP43 in a cell comprising contacting the cell with an oligonucleotide complementary to SEQ ID NO: 1, or a fragment thereof, whereby the oligonucleotide inhibits expression of the protein.

In another aspect, the invention provides a method of treating a subject having a viral infection by administering to the subject an effective amount of a compound that inhibits the expression or activity of UBP43, thereby treating the subject.

In one embodiment, the viral infection is an HIV infection. In another embodiment, the viral infection is filovirus.

In another embodiment, the activity of UBP43 is modulated by a molecule selected from the group consisting of a small molecule, a peptide, a peptide mimetic, a lipid, and an antibody. In another embodiment, the expression of UBP43 is modulated by siRNA, a ribozyme, or antisense RNA.

In specific embodiments, the virus is drug sensitive or drug resistant.

In another aspect, the invention provides a method of treating a subject having an HIV infection by administering to the subject an effective amount of a compound that inhibits the expression or activity of UBP43, thereby treating the subject.

In another embodiment, the activity of UBP43 is modulated by a molecule selected from the group consisting of a small molecule, a peptide, a peptide mimetic, a lipid, and an antibody. In another embodiment, the expression of UBP43 is modulated by siRNA, a ribozyme, or antisense RNA.

In specific embodiments, the virus is drug sensitive or drug resistant.

In another aspect, the invention provides a method of treating a subject having a viral infection by administering to the subject an effective amount of a composition that increases the expression or activity of UBEL-1, thereby treating the subject.

In one embodiment, the viral infection is an HIV infection. In another embodiment, the viral infection is filovirus infection.

In a related embodiment, the composition is a gene therapy vector.

In specific embodiments, the virus is drug sensitive or drug resistant.

In another aspect, the invention provides a method of treating a subject having an HIV infection by administering to the subject an effective amount of a composition that increases the expression or activity of UBEL-1, thereby treating the subject. In a related embodiment, the composition is a gene therapy vector.

In specific embodiments, the virus is drug sensitive or drug resistant.

In another aspect, the instant invention provides a pharmaceutical composition comprising a UBP43 inhibitor and a pharmaceutically acceptable carrier. In a related embodiment, the pharmaceutical composition is for the treatment of viral infection.

In another aspect, the instant invention provides a vector comprising SEQ ID NO:3 for use in gene therapy. In a related embodiment, the vector is for the treatment or prevention of viral infection, e.g., HIV infection.

### BRIEF DESCRIPTION OF THE FIGURES

FIG. 1 depicts a graph indicating that coexpression of HIV(NL43), ISG15 and UBEL-1 overexpression fully inhibit HIV-1 replication, whereas ISG15, or UBEL-1 alone only partially inhibit HI-1 replication.

FIGS. 2A-C depict western blots, and a quantitation thereof, indicating that ISG15 inhibits ubiquitination of Gag. FIG. 2A depicts a western blot with anti-gag antibody.

FIG. 2B depicts a western blot with anti-ubiquitin antibody. FIG. 2C depicts a bar graph that quantitates the western blot of FIG. 2C. The lanes in the western blot are: 1:Gag+Ub. 2:dGag+Ub, 3:Gag+ISG15+Ub, 4:Gag+UBE1L+Ub, and 5:Gag+ISG15+UBE1L+Ub.

FIG. 3A-D depict western blots indicating that ISG15 inhibits the interaction between Gag and Tsg101. FIG. 3A depicts a western blot of MG132 untreated cells immunoprecipitated using anti-tag101 and immunoblotted using anti-gag. FIG. 3B depicts a western blot of MG132 untreated cells using anti-Tsg101 antibodies. In FIGS. 3A and B, lanes 1-7 contain: 1: Control, 2: NL43, 3: NL43+TSG101HA, 4: NL43+TSG101HA+ISG15His, 5: NL43+TSG101HA+ISG15His+UBE1L, 6: NL43+TSG101HA+ISG15His, and 7: NIL43+TSG101HA+UB43. FIG. 3C depicts a western blot of MG132 treated cells immunoprecipitated using anti-tag101 and immunoblotted using anti-gag. FIG. 3D depticts a western blot of MG132 untreated cells using anti-Tsg101 antibodies. In FIGS. 3C and D, lanes 1-7 contain: 1: Control, 2: NIA3, 3: NL43+TSG101HA, 4: NL43+TSG101HA+ISG15His 5: NL43+TSG101HA+ISG15His+UBE1L, 6: NL43+TSG101HA+UB43, and 7:NL43+TSG101HA+ISG15His+UB43.

FIGS. 4A-B depict western blots indicating that ISG15 inhibits ubiquitination of TSG101. FIG. 4A depicts a western blot of cell lysates using anti-HA. FIG. 4B depicts a western blot of immunoprecipitated cell lystates using anti-Tsg101 and immunoblotted using anti-HA. Lanes 1-7 contain: 1:Control, 2:Tsg101, 3:Tsg101+Ub, 4:Tsg101+ISG15+Ub, 5:Tsg101+ISG15+Ub+UBE1L, 6:Tsg101+ISG15, and 7:Tsg101+ISG15+UBE1L.

FIG. 5 depicts a graph indicating that ISG15 inhibits HIV release in U.1.1 cells.

FIG. 6 depicts a western blot demonstrating the expression of ISG15 in lentivirus transduced U.1.1 cells.

FIGS. 7A-B depicts the sequence of UBP43. FIG. 7A depicts the nucleic acid sequence of human UBP43 (SEQ ID NO: 1) and FIG. 7B depicts the polypeptide sequence of UBP43 (SEQ ID NO:2).

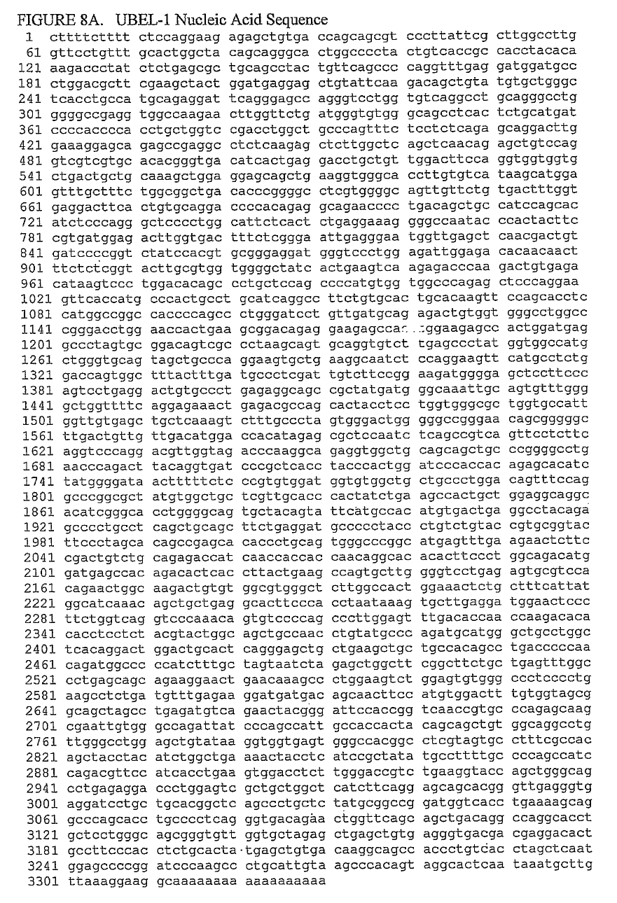

FIGS. 8A-B depicts the sequence of UBEL-1. FIG. 8A depicts the nucleic acid sequence of human UBP43 (SEQ ID NO:3) and FIG. 8B depicts the polypeptide sequence of UBP43 (SEQ ID NO:4).

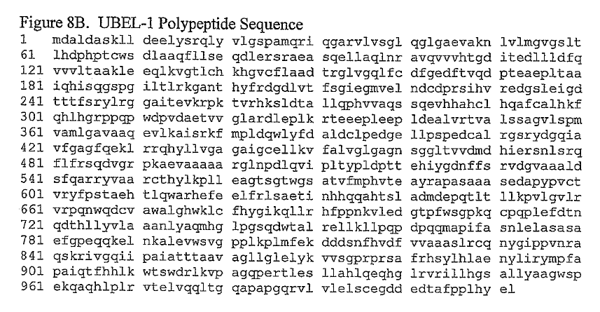

FIGS. 9A-B depicts the sequence of ISG15. FIG. 9A depicts the nucleic acid sequence of human UBP43 (SEQ ID NO:5) and FIG. 9B depicts the polypeptide sequence of UBP43 (SEQ ID NO:6).

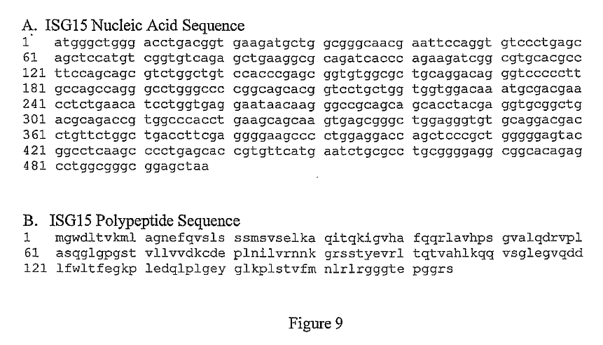

### DETAILED DESCRIPTION OF THE INVENTION

The present invention is based, at least in part, on the discovery that interferon induced ISG15 polypeptides modulate the cellular ubiquitin regulation pathway used by viruses in their replication cycle i.e., the conjugation and deconjugation of ISG15 from cellular proteins, and, therefore, modulate the replication of viruses that use components of this pathway for replication. Exemplary polypeptides that modulate the activity of ISG15 conjugation are UBP43 and UBEL-1. Accordingly, the instant invention provides novel targets and methods for the prevention and treatment of viral infection, e.g., HIV infection.

The term “ubiquitination pathway” is intended to include proteins that are responsible for conjugating or deconjugating an ubiquitin molecule, or a ubiquitin-like polypeptide such as ISG15, to cellular proteins or a protein that regulates the activity or expression of a protein that regulates the activity of a protein that is responsible for conjugating or deconjugating an ubiquitin molecule, or a ubiquitin-like polypeptide such as ISG15, to cellular proteins. Exemplary proteins involved in the ubiquitin pathway include UBP43 and UBEL-1. The conjugation of ISG15 to cellular proteins has also referred to as ISGylation.

### Screening Methods

The invention provides methods or screening assays for identifying modulators of the deubiquitination pathway, e.g., modulators of the activity or expression of UBP43 and UBEL-1. These modulators include compounds or agents (e.g., peptides, peptidomimetics, peptoids, polynucleotides, small molecules, lipids, or other drugs) which (a) bind to a polypeptide involved in the deubiquitination pathways; (b) have a modulatory (e.g., up-regulatory, inductive; potentiating, stimulatory, down-regulatory, suppressive or inhibitory) effect on the activity of a polypeptide involved in the deubiquitination pathways; (c) have a modulatory effect on the interactions of a polypeptide involved in the deubiquitination pathways with one or more of its substrates; or (d) have a modulatory effect on the expression of a polypeptide involved in the deubiquitination pathways. An exemplary assay comprises contacting a compound of interest with cells and comparing the expression of a polypeptide involved in the deubiquitination pathway in the cells before and after said contact.

In many embodiments, the compounds of interest are small molecules or biomolecules. Small molecules include, but are not limited to, inorganic molecules and small organic molecules. Biomolecules include, but are not limited to, naturally-occurring and synthetic compounds that have a bioactivity in mammals, such as polypeptides, polysaccharides, and polynucleotides. One of ordinary skill in the art will appreciate that the nature of a compound of interest may vary depending on the nature of the protein encoded by the polypeptide involved in the deubiquitination pathway, e.g., UBP43 or UBEL-1, being investigated.

The compounds to be tested can be obtained from numerous sources, including systematic libraries of natural and/or synthetic compounds. Test compounds can also be obtained from combinatorial libraries that are constructed according to any method known in the art. Suitable libraries include biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem., 37:2678-85, 1994); spatially addressable parallel solid phase or solution phase libraries; libraries made by synthetic library methods requiring deconvolution; libraries made by the “one-bead one-compound” library method; and libraries made by synthetic library methods using affinity chromatography selection. See, e.g., Lam, Anticancer Drug Des., 12:145, 1997.

Nucleic acid molecules that encode polypeptides involved in the deubiquitination pathway can be inserted into gene delivery vectors and used as gene therapy vectors. As used herein, the term “vector” refers to a nucleic acid molecule capable of transporting a cloned gene or its cDNA to which it has been linked and can include a plasmid, cosmid or viral vector. The vector can be capable of autonomous replication or it can integrate into a host DNA. Viral vectors include, e.g., replication defective retroviruses, lentiviruses, adenoviruses and adeno-associated viruses.

Gene therapy vectors can be delivered to a subject by, for example, intravenous administration, intraportal administration, intrabiliary administration, intra-arterial administration, direct injection into the liver parenchyma, by intramusclular injection, by inhalation, by perfusion, or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Nucleic acid molecules encoding, for example, UBEL-1 can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

In addition, numerous gene therapy methods that take advantage of retroviral vectors, for treating a wide variety of diseases are well-known in the art (see, e.g., U.S. Pat. Nos. 4,405,712 and 4,650,764; Friedmann, 1989, Science, 244:1275-1281; Mulligan, 1993, Science, 260:926-932, R. Crystal, 1995, Science 270:404-410, each of which are incorporated herein by reference in their entirety). Furthermore expression of a gene in a retroviral or lentivirus vector can be selectively targeted to virus infected cells, by using virus inducible promoter such as HIV-LTR (see, e.g., Bednarik D. et al. (1989) Proc Natl Acad. Sci. 86:4958-62; Su, Y. et al (1995) J. Virology 69:110-21; and Dropulic. B. et al. (1996) Proc Natl Acad. Sci. 93:11103-8 all of which are incorporated herein by reference in their entirety).

An increasing number of these methods are currently being applied in human clinical trials (Morgan, R., 1993, BioPharm, 6(1):32-35; see also The Development of Human Gene Therapy, Theodore Friedmann, Ed., Cold Spring Harbor Laboratory Press, Cold Spring Harbor, N.Y., 1999. ISBN 0-87969-528-5, which is incorporated herein by reference in its entirety). The safety of these currently available gene therapy protocols can be substantially increased by using retroviral vectors of the present invention. For example, where the retroviral vector infects a non-targeted cell, the retroviral genome will integrate but will not be transcribed. However, when the retroviral vector containing a tissue, or cell, specific regulatory element infects a targeted cell the active tissue specific promoter will result in transcription and translation of the viral genome. The pharmaceutical compositions can be included in a container, pack, or dispenser together with instructions for administration.

An “antisense” polynucleotide comprises a nucleotide sequence which is complementary to a “sense” polynucleotide, e.g., complementary to the coding strand of a double-stranded cDNA molecule or to an mRNA sequence. An antisense polynucleotide can be complementary to the entire coding strand of a nucleic acid molecule which encodes a polypeptide involved in the deubiquitination pathway, e.g., UBP43, or a portion thereof. Alternatively, the antisense polynucleotide can be highly identical to a complementary portion of a “sense” polynucleotide, e.g., 85%, 90%, 95%, or 98% identical.

In many embodiments, an antisense polynucleotide of the present invention includes at least about 10, 15, 20, 25, 30, 35, 40, 45, 50 or more nucleotides. An antisense polynucleotide can be designed according to the rules of Watson and Crick base pairing. In one embodiment, an antisense polynucleotide is chemically synthesized using naturally occurring nucleotides or variously modified nucleotides designed to increase the biological stability of the molecules or to increase the physical stability of the duplex formed between the antisense and sense polynucleotides. Examples of modified nucleotides which can be used to generate an antisense polynucleotide include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil-1, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. In another embodiment, an antisense polynucleotide of the present invention is produced biologically by using an expression vector into which the target sequence is subcloned in an antisense orientation.

In many cases, the antisense polynucleotide molecules of the invention are administered to a subject or generated in situ such that they hybridize or bind to cellular mRNA and/or genomic DNA encoding a nucleic acid encoding a polypeptide involved in the deubiquitination pathway, thereby inhibiting the expression of the corresponding polypeptide involved in the deubiquitination pathway. An exemplary route of administration of antisense polynucleotide molecules is direct injection at a tissue site (e.g., intestine). In one embodiment, antisense polynucleotide molecules are modified to target selected cells and then administered systemically. For systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intracellular concentrations of the antisense molecules, vector constructs in which the antisense polynucleotide molecule is placed under the control of a strong promoter, such as pol II or pol III promoter, may be employed.

In yet another embodiment, an antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other (Gaultier et al., Polynucleotides. Res., 15:6625-6641, 1987). The antisense polynucleotide molecule can also comprise a 2′-o-methylribonucleotide or a chimeric RNA-DNA analogue.

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes) can be used to catalytically cleave mRNA transcripts of the nucleic acid molecule that encodes a polypeptide involved in the deubiquitination pathway to thereby inhibit translation of said mRNA. A ribozyme having specificity for a target can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a protein-encoding mRNA. Alternatively, mRNA transcribed from a gene of the invention can be used to select a catalytic RNA having a specific ribonuclease activity from a pool of RNA molecules. Alternatively, expression of a protein can be inhibited by targeting the regulatory region of these genes (e.g., the promoter and/or enhancers) with complementary nucleotide sequences that will form triple helical structures with the target sequence to prevent transcription of the gene in target cells.

Expression of a polypeptide involved in the deubiquitination pathway can also be inhibited by using RNA interference (“RNAᵢ”). Sequences capable of inhibiting gene expression by RNA interference can have any desired length. For instance, the sequence can have at least 15, 20, 25, or more consecutive nucleotides. The sequence can be dsRNA or any other type of polynucleotide, provided that the sequence can form a functional silencing complex to degrade the target mRNA transcript.

In one embodiment, the sequence comprises or consists of a short interfering RNA (siRNA). The siRNA can be, without limitation, dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA may take place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′ dinucleotide overhang, such as UU, can be used for siRNA design. In some cases, G residues in the overhang are avoided because of the potential for the 5 siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA is designed to have two inverted repeats separated by a short spacer sequence. In some examples, the siRNA end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see e.g., Sui et al, Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (Lee et al, Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one example, the 5′ overhang includes about 6 nucleotide residues. In still yet another embodiment, the target sequences for RNAi are 21-mer or 20-mer sequence fragments selected from coding sequences, such as SEQ ID NO: 1. The target sequences can be selected from either ORF regions or non-ORF regions. The 5′ end of each target sequence has dinucleotide “NA,” where “N” can be any base and “A” represents adenine. The remaining 19-mer or 18-mer sequence has a GC content of between 30% and 65%. In many examples, the remaining 19-mer or 18-mer sequence does not include any four consecutive A or T (i.e., AAAA or TTTT), three consecutive G or C (i.e., GGG or CCC), or seven “GC” in a row.

Additional criteria can be used for RNAi target sequence design. In one example, the GC content of the remaining 19-mer or 18-mer sequence is limited to between 35% and 55%, and any 19-mer or 18-mer sequence having three consecutive A or T (i.e., AAA or TTT) or a palindrome sequence with 5 or more bases is excluded. In addition, the 19-mer or 18-mer sequence can be selected to have low sequence homology to other human genes. In one embodiment, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer/18-mer sequences producing no hit to other human genes under the BLASTN search can be selected. During the search, the e-value may be set at a stringent value (such as “1”). Furthermore, the target sequence can be selected from the ORF region, and is at least 75-bp from the start and stop codons.

The effectiveness of the siRNA sequences can be evaluated using various methods known in the art. For instance, a siRNA sequence of the present invention can be introduced into a cell. The polypeptide or mRNA level of a polypeptide involved in the deubiquitination pathway, e.g., UBP43 in the cell can be detected. A substantial change in the expression level of the protein before and after the introduction of the siRNA sequence is indicative of the effectiveness of the siRNA sequence in suppressing the expression of the target. In one example, the expression levels of other genes are also monitored before and after the introduction of the siRNA sequence. A siRNA sequence which has inhibitory effect on expression but does not significantly affect the expression of other genes can be selected. In another example, multiple siRNA or other RNAi sequences can be introduced into the same target cell. These siRNA or RNAi sequences specifically inhibit the gene expression but not the expression of other genes.

The invention provides methods for screening for inhibitors of a polypeptide involved in the deubiquitination pathway, e.g., UBP43. An exemplary screening method includes contacting each aliquot of a sample with one of a plurality of test compounds, and comparing expression of UBP43 in each of the aliquots to determine whether any of the test compounds substantially decreases the level of expression or activity of UBP43, e.g., the regulation of ISG15 deconjugation, relative to samples contacted with other test compounds or relative to an untreated sample or control sample. In addition, the screening methods can include directly contacting a test compound with a protein and then determining the effect of the compound on the activity of the protein.

Moreover, the invention provides methods for screening compounds capable of modulating the binding between a UBP43 and a binding partner, e.g., a polypeptide involved in the deubiquitination pathway such as ISG15. The compounds may be either small molecules or biomolecules. The compounds can be obtained from a variety of libraries well-known in the art.

Modulators of the expression or protein activity of UBP43 are useful as therapeutic agents for treating viral infections. Such modulators (e.g., antagonists or agonists) may be formulated as pharmaceutical compositions, as described herein.

Other methods for screening for protein inhibitors are described in U.S. Pat. Nos. 4,980,281, 5,266,464, 5,688,635, and 5,877,007, all of which are incorporated herein by reference.

In another embodiment, antibodies, or fragments thereof, can be screened for modulatory effects. Antibodies and T-cell antigen receptors (TCR) which immunospecifically bind a polypeptide, polypeptide fragment, of, e.g., SEQ ID NOs:2 can be used in the methods of the instant invention. Antibodies used in the methods of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above.

Preferred antibodies of the invention are those the bind to, for example, UBP43 and block the deubiquitionation of ISG15. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Nab) is meant to include intact molecules, as well as, antibody fragments (such as, for example, Fab and F(ab′).sub.2 fragments) which are capable of specifically binding to protein. Fab and F(ab′).sub.2 fragments lack the Fc fragment of intact antibody, clear more rapidly from the circulation of the animal or plant, and may have less non-specific tissue binding than an intact antibody (Wahl et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies used in the methods of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies used in the methods of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies used in the methods of the present invention may be described or specified in terms of the epitope(s) or portion(s) of a polypeptide of the present invention which they recognize or specifically bind.

Antibodies used in the methods of the present invention may also be described or specified in terms of their cross-reactivity. Antibodies that do not bind any other analog, ortholog, or homologue of a polypeptides described herein are included. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6M, 5×10-7 M, 107 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

Antibodies used in the methods of the present invention may act as agonists or antagonists of the polypeptides described herein. For example, the present invention includes antibodies which disrupt interactions with the polypeptides described herein partially or fully, e.g., the interaction between ISG15 and UBP43.

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

In order to screen the compounds described above, the following assays for deubiquitinating enzyme activity may be used. Assays that are commonly known are described in, for example, Zhu et al. (1997) Journal of Biological Chemistry 272:51-57, Mitch et al. (1999) American Journal of Physiology 276:C1132-C1138, Liu et al. (1999) Molecular and Cell Biology 19:3029-3038, and such as those cited in various reviews, for example, Ciechanover et al. (1994) The FASEB Journal 8:182-192, Chiechanover (1994) Biol. Chem. Hoppe-Seyler 375:565-581, Hershko et al. (1998) Annual Review of Biochemistry 67:425-479, Swartz (1999) Annual Review of Medicine 50:57-74, Ciechanover (1998) EMBO Journal 17:7151-7160, and D'Andrea et al. (1998) Critical Reviews in Biochemistry and Molecular Biology 33:337-352. These assays include, but are not limited to, the disappearance of substrate, including decrease in the amount of ubiquitinated/ISG15 substrate protein or protein remnant, appearance of intermediate and end products, such as appearance of free ubiquitin/ISG15 monomers, general protein turnover, specific protein turnover, ubiquitin binding, binding to ubiquitinated/ISG15 substrate protein, subunit interaction, interaction with ATP, interaction with cellular components such as trans-acting regulatory factors, stabilization of specific proteins, and the like.

### Pharmaceutical Compositions and Kits

The invention is further provides pharmaceutical compositions for treating or preventing viral infections. In one embodiment, a pharmaceutical composition of the present invention includes a modulator of the expression or activity of a polypeptide involved in the deubiquitination pathway, e.g., UBEL-1 or UBP43. In another embodiment, a pharmaceutical composition of the present invention includes an antibody specific for a polypeptide involved in the deubiquitination pathway, or an antisense or RNAi sequence of the nucleic acid molecule that encodes a polypeptide involved in the deubiquitination pathway.

The modulators, antibodies, antisense or RNAi sequences, or other biologically active agents of the present invention can be formulated into a pharmaceutical composition. A typical pharmaceutical composition includes a pharmaceutically acceptable carrier which may include any solvent, solubilizer, filler, stabilizer, binder, absorbent, base, buffering agent, lubricant, controlled release vehicle, diluent, emulsifying agent, humectant, lubricant, dispersion media, coating, antibacterial or antifungal agent, isotonic or absorption delaying agent that is compatible with pharmaceutical administration. The use of such media and agents for pharmaceutically active substances is well-known in the art. Methods for preparing a pharmaceutical composition of an active agent are well known in the art.

A pharmaceutical composition of the invention is formulated to be compatible with its intended route of administration. Examples of routes of administration include parenteral, intravenous, intradermal, subcutaneous, oral, inhalative, transdermal topical, transmucosal, or rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide: The parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, but are not limited to, physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. In many embodiments, the composition is stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is desirable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. In many examples, dispersions are prepared by incorporating the active compound into a sterile vehicle which contains a basic dispersion medium and the required other ingredients from those enumerated above. In the case of sterile powders for the preparation of sterile injectable solutions, example methods of preparation include vacuum drying or freeze-drying which yields a powder of the active ingredient plus any additional desired ingredient from a previously sterile-filtered solution thereof.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from a pressured container or dispenser which contains a suitable propellant, e.g., a gas such as carbon dioxide, or a nebulizer.

Systemic administration may be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, a bioactive agent may be formulated into ointments, salves, gels, or creams as generally known in the art.

A bioactive agent may also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

In one embodiment, oral or parenteral compositions are formulated in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In many embodiments, compounds which exhibit large therapeutic indices are selected. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In many instances, the dosage of such compounds lies within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The invention further provides kits for use in treating a viral infection. For example, the kit can comprise a compound or agent capable of modulating the activity of a polypeptide that regulates ISG15, e.g., UBP43 or UBEL-1. The compound can be packaged in a suitable container. The kit can further comprise instructions for using the kit to treat or prevent viral infection.

For example, in one embodiment, the kit comprises a pharmaceutical composition containing an effective amount of an inhibitor of UBP43 and instruction for use in treating or preventing viral infection. In another embodiment, the kit comprises a pharmaceutical composition comprising an effective amount of a compound that stimulates that expression or activity of UBEL-1.

### Methods of Treatment

A further embodiment of the present invention embraces the administration of a pharmaceutical composition, in conjunction with a pharmaceutically acceptable carrier, diluent, or excipient, to treat a subject having, or at risk of having, a viral infection, e.g., an HIV infection. The instant invention provides methods of treatment and prevention of viral infection in a subject, e.g., a mammal such as a human.

The methods of the invention comprise administering to a subject an effective amount of a compound, or pharmaceutical composition, that regulates that activity of a polypeptide that regulates ISG15 conjugation or deconjugation. By increasing the conjugation of ISG15, or decreasing the deconjugation of ISG15, viral replication is inhibited. In one embodiment, the methods of treatment of the instant invention comprise administering to a subject an effective amount of a compound that inhibits ISG15 deconjugation by, for example, inhibiting the activity or expression of UBP43. Alternatively, the methods of treatment of the instant invention comprise administering to a subject an effective amount of a compound that enhances the level of ISG15 conjugation by, for example, enhancing the activity or expression of UBEL-1.

Pharmaceutical compositions suitable for use in the present invention include compositions wherein the active ingredients are contained in an effective amount to achieve the intended purpose, e.g., treatment or prevention of viral infections. The determination of an effective dose or amount is well within the capability of those skilled in the art. For any compound, the therapeutically effective dose can be estimated initially either in cell culture assays, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, polynucleotide, polypeptide, or fragments thereof, antibodies, lipids, agonists, antagonists or inhibitors, which ameliorates, reduces, diminishes, or eliminates the symptoms or condition. Therapeutic efficacy and toxicity can be determined by standard pharmaceutical procedures in cell cultures or in experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, the sensitivity of the patient, and the route of administration.

The practitioner, who will consider the factors related to an individual requiring treatment, will determine the exact dosage. Dosage and administration are adjusted to provide sufficient levels of the active component, or to maintain the desired effect. Factors which may be taken into account include the severity of the individual's disease state; the general health of the patient; the age, weight, and gender of the patient; diet; time and frequency of administration; drug combination(s); reaction sensitivities; and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

As a guide, normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors or activators. Similarly, the delivery of polynucleotides or polypeptides will be specific to particular cells, conditions, locations, and the like.

The invention is further illustrated by the following examples, which should not be construed as limiting.

### EXAMPLES

### Example 1

### ISG15 inhibits HIV-1 Replication

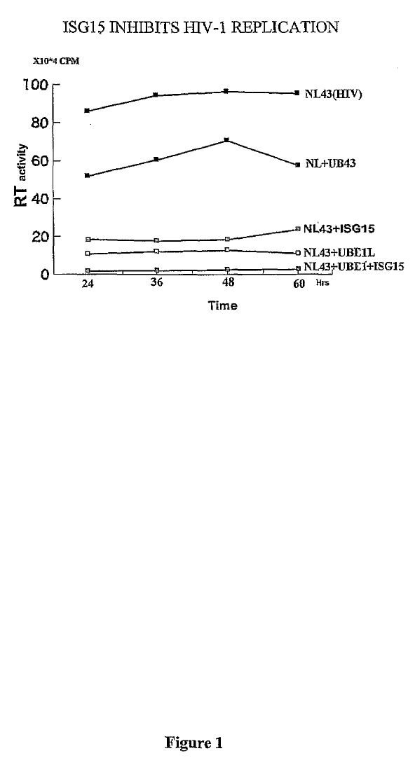

It has previously been observed that IFNα mediated inhibition of HIV-1 replication correlates with the induction of ISG15 and that the inhibition occurs at the level of virus assembly and budding. Addressing the molecular mechanism of this inhibition, it has been determined that over-expression of ISG15 inhibits the HIV-1 replication, and further inhibition was observed in cells over expressing the ISG15 activating ligase UBEL-1 (see FIG. 1). The ISG15 deconjugating enzyme UBP43 releases ISG15 mediated inhibition. Overexpression of ISG15 inhibited ubiquitination of cellular proteins as well as ubiquitinated proteins present in HIV-1 virons.

### Example 2

### ISG15 Inhibits Ubiquitination of Gag

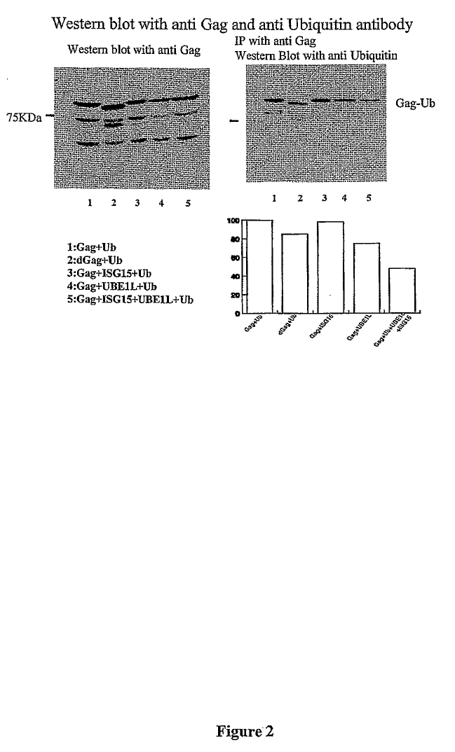

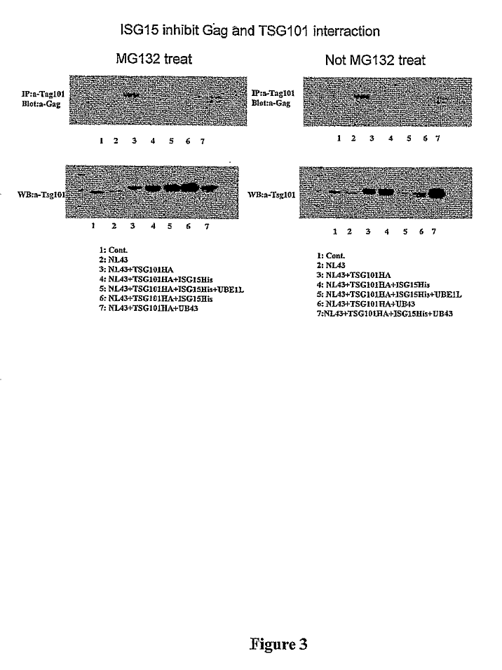

Since monoubiquination of Gag was shown to be important for the HIV-1 budding process, it was next examined as to whether ISG15 can inhibit mono-ubiquination of the Gag protein. Overexpression of ubiquitin, ISG15, or UBEL-1 did not affect relative levels of ectopic Gag protein in transfected cells, indicating that neither ubiquitin nor ISG15 induced Gag degradation. However, ubiquitination of Gag polypeptides was inhibited in cells that express UBEL-1 and ectopic ISG15. Cells were co-transfected with plasmids expressing HIV-1 gag alone or in the presence of UBEL-1 and ISG15. Cell lysates were analyzed 24 hrs post tranfection for the presence of Gag and ubiquitinated gag. dGag represents mutant of Gag with deleted p6 region. The results show modest inhibition of Gag ubiqutination by ISG15 (see FIG. 2).

### Example 3

### ISG15 Inhibits Interaction between Gag and Tsg101

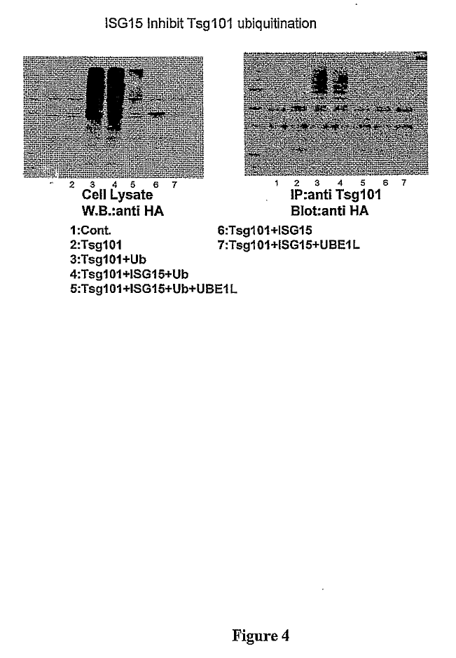

Cells were co-transfected with Gag and Tsg101 expressing plasmid alone or in the presence of ISG15 or ISG15 and UBEL-1. 24 hr post transfection cell lysates were immune precipitated with HA antibodies (detecting Tsg101) and precipitates immune blotted with Gag antibodies (see FIG. 3). The relative levels of Tsg101 in the precipitates was estimated by immune blotting with HA antibodies. The experiments were done in the presence and absence of MG132 that inhibits proteosome degradation. The results indicate binding of Tsg101 with Gag (lane 3) that is inhibited in the presence of ISG1 (lanes 4, 5, 6).

### Example 4

### ISG15 Inhibits Ubiquitination of Tsg101

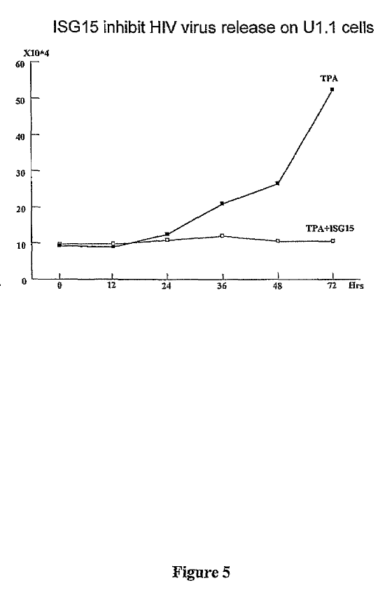

In order to determine if ISG15 inhibits ubiquitionation of Tsg101, cells were transfected with Tsg101 alone or in the presence of ubiquitin, ISG15 and UBEL-1 as indicated. Cells lysates were analyzed 24 for the presence of ubiquitinylated proteins with Ha antibodies (left side) or immune precipitated with Tsg101 antibodies and the ubiquitinalete Tsg101 detected by immune blotting with HA antibodies (see, FIG. 4). The results show that in the presence of ISG15 ubiquitination of Tsg101 is inhibited.

### Example 5

### ISG15 Inhibits Expression of the Integrated HIV-1 Provirus

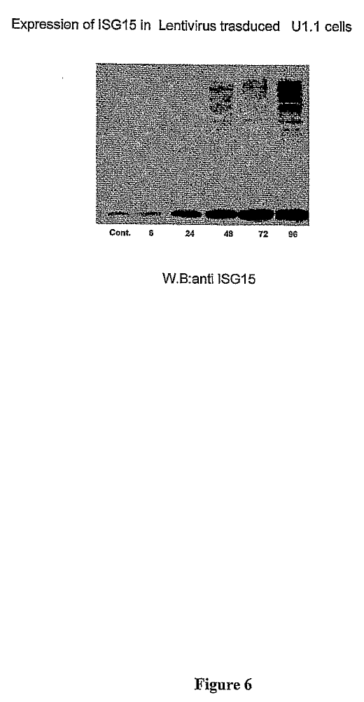

In order to determine if ISG15 inhibits expression of the integrated HIV-1 provirus, U.1.1 cells that contain a single copy of an integrated HIV-1 provirus were transduced by lentivirus expressing ISG15. Transduced cells and untranduced parental cells were then treated with TPA that induces expression of the integrated provirus and the levels of HIV-1 virus in the medium were measure by the reverse transcriptase assay at indicated time (see FIG. 5). The results have shown that while the viral particles were released from TPA treated parental line virus release was blocked in the line expressing ISG15.

### Example 6

### Expression of ISG15 in Lentivirus Transduced U.1.1 Cells

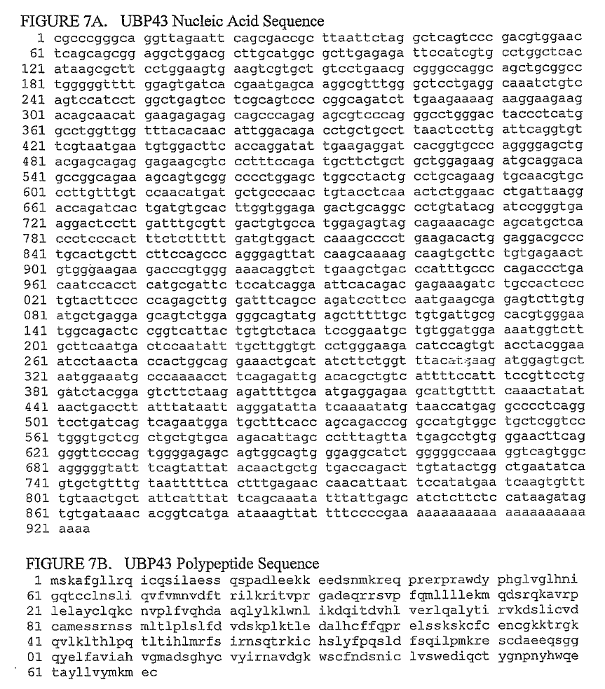

In order to determine the effect of expression of ISG15 in lentivirus transduced U.1.1 cells, the lysates from ISG15 transduced cells were isolated at different times post transduction and immune blotted with the anti ISG15 antibodies (see FIG. 6). The results show that conjugation of ISG15 to cellular proteins can be detected at 48 hrs post transduction while low levels of conjugation can be detected as early as 24 hr post transduction.

### Incorporation by Reference

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of inhibiting the viral replication comprising;
contacting a cell infected with a virus with a compound that modulates the activity or expression of a polypeptide involved in the ubiquitination pathway used by the virus during viral replication;
thereby inhibiting the replication of the virus.

2. The method of claim 1, wherein modulation of the activity or expression of a polypeptide involved in the ubiquitination pathway leads to increased conjugation of ISG15 to cellular proteins involved in viral replication.

3. The method of claim 1, wherein the modulation is upregulated.

4. The method of claim 1, wherein the modulation is down-regulated.

5. The method of claim 1, wherein the virus is selected from the group consisting of retroviruses and Class A viruses.

6. The method of claim 5, wherein the retrovirus is selected from the group consisting of human immunodeficiency virus, human T-cell leukemia virus type 1, and human T-cell leukemia virus type 2.

7. The method of claim 5, wherein the Class A virus is filovirus.

8. The method of claim 1, wherein the activity is modulated by a molecule selected from the group consisting of a small molecule, a peptide, a peptide mimetic, a lipid, and an antibody.

9. The method of claim 1, where the expression is modulated by siRNA, a ribozyme, or antisense RNA.

10. The method of claim 1, wherein the virus is drug sensitive.

11. The method of claim 1, wherein the virus is drug resistant.

12. A method of inhibiting the replication of viruses that use the cellular endosomal pathways to replicate, comprising:
contacting a cell infected with the virus with a compound capable of inhibiting the activity or expression of UBP43;
thereby inhibiting viral replication.

13. The method of claim 12, wherein inhibition of the expression or activity of UBP43 leads to inhibition of the deconjugation of ISG15 from proteins involved in HIV replication.

14. The method of claim 12, wherein the virus is selected from the group consisting of retroviruses and Class A viruses.

15. The method of claim 14, wherein the retrovirus is selected from the group consisting of human immunodeficiency virus, human T-cell leukemia virus type 1, and human T-cell leukemia virus type 2.

16. The method of claim 14, wherein the Class A virus is filovirus (Ebola virus).

17. The method of claim 12, wherein the activity of UBP43 is inhibited by a molecule selected from the group consisting of a small molecule, a peptide, a peptide mimetic, a lipid, and an antibody.

18. The method of claim 12, where the expression of UBP43 is inhibited by siRNA, a ribozyme, or antisense RNA.

19. The method of claim 12, wherein the virus is drug sensitive.

20. The method of claim 12, wherein the virus is drug resistant.

21. A method of inhibiting the replication of HIV comprising:
contacting a cell infected with HIV with a compound capable of inhibiting the activity or expression of UBP43;
thereby inhibiting HIV replication.

22-24. (canceled)

25. A method of inhibiting the replication of viruses that use the cellular endosomal pathways to replicate, comprising:
contacting a cell infected with the virus with a compound capable of stimulating the expression or activity of UBEL-1;
thereby inhibiting viral replication.

26-29. (canceled)

30. A method of inhibiting the expression of UBP43 in a cell comprising contacting the cell with an oligonucleotide complementary to SEQ ID NO:1, or a fragment thereof, whereby the oligonucleotide inhibits expression of the protein.

31. A method of treating a subject having a viral infection comprising;
administering to the subject an effective amount of a compound that inhibits the expression or activity of UBP43;
thereby treating the subject.

32-43. (canceled)

43. A method of treating a subject having a viral infection comprising;
administering to the subject an effective amount of a composition that increases the expression or activity of UBEL-1;
thereby treating the subject.

44. The method of claim 43, wherein the viral infection is an HIV infection.

45. The method of claim 43, wherein the viral infection is filovirus (Ebola virus).

46-52. (canceled)

53. A pharmaceutical composition comprising a UBP43 inhibitor and a pharmaceutically acceptable carrier.

54. (canceled)

55. A vector comprising SEQ ID NO:3 for use in gene therapy.

56. (canceled)
